(19) United States
(12) Patent Application Publication
(10) Pub. No.: US 2023/0277823 A1
(43) Pub. Date: Sep. 7, 2023

(54) LUMEN INSERTABLE CAPSULE

(71) Applicant: Novo Nordisk A/S, Bagsvaerd (DK)

(72) Inventors: Brian Mouridsen, Fredensborg (DK); Jacob Pyung Hwa Jepsen, Fredensborg (DK); Nikolaj Eusebius Jakobsen, Soeborg (DK); Drago Sticker, Malmoe (SE); Aghiad Ghazal, Koebenhavn SV (DK)

(21) Appl. No.: 18/019,148

(22) PCT Filed: Aug. 5, 2021
(86) PCT No.: PCT/EP2021/071857

§ 371 Date: Feb. 1, 2023

(30) Foreign Application Priority Data
Aug. 8, 2020 (EP) 20190168.3

(51) Int. Cl. A61M 31/00; A61K 9/00

(52) U.S. Cl. CPC A61M 31/002; A61K 9/0004

(57) ABSTRACT

A capsule (100) suitable for insertion into a gastrointestinal lumen. The capsule (100) comprises: a capsule housing (110, 120), a reservoir (A) accommodating a drug substance, a drug outlet (190), an actuation chamber (118), a movable separator (160) between the actuation chamber (118) and the reservoir (A), and a drive system comprising a gas expansion unit (150) and a trigger (145, 140, 170). The gas expansion unit (150) comprises a gas gate (151) and the trigger (145, 140, 170) comprises a trigger member (170) and a swellable portion (140) comprising sponge material, wherein wetting of the sponge material causes swelling of the sponge material thereby causing relative movement between the gas expansion unit (150) and the trigger member (170) to open the gas gate (151). Pressurized gas flows from them to the actuation chamber (118) thereby exerting load on the separator (160) for expelling the drug substance.

The present invention relates to lumen insertable devices, such as ingestible capsules for delivery of a drug substance to a subject user.

### BACKGROUND OF THE INVENTION

In the disclosure of the present invention reference is mostly made to the treatment of diabetes by delivery of insulin, however, this is only an exemplary use of the present invention.

May people suffer from diseases, such as diabetes, which requires them to receive injections of drugs on a regular and often daily basis. To treat their disease these people are required to perform different tasks which may be considered complicated and may be experienced as uncomfortable. Furthermore, it requires them to bring injection devices, needles and drugs with them when they leave home. It would therefore be considered a significant improvement of the treatment of such diseases if treatment could be based on oral intake of tablets or capsules.

However, such solutions are very difficult to realise, since protein-based drugs will be degraded and digested rather than absorbed when ingested.

To provide a working solution for delivering insulin into the bloodstream through oral intake, the drug has to be delivered firstly into a lumen of the gastrointestinal tract and further into the wall of the gastrointestinal tract (lumen wall). This presents several challenges among which are: (1) The drug has to be protected from degradation or digestion by the acid in the stomach. (2) The drug has to be released while being in the stomach, or in the lower gastrointestinal tract, i.e. after the stomach, which limits the window of opportunity for drug release. (3) The drug has to be delivered at the lumen wall to limit the time exposed to the degrading environment of the fluids in the stomach and in the lower gastrointestinal tract. If not released at the wall, the drug may be degraded during its travel from point of release to the wall or may pass through the lower gastrointestinal tract without being absorbed, unless being protected against the decomposing fluids.

Capsule devices have been proposed for delivery of a drug substance into a lumen or lumen wall. After insertion of the capsule, such as by swallowing the capsule into the GI system of the subject, drug delivery should be performed but initiated only upon a pre-defined condition is met. Triggering systems for triggering of an expelling mechanism typically rely on mechanical energy for providing the triggering movement for actuation of a drive system.

WO 2018/049133 includes disclosure of various different ingestible devices wherein some of these include an osmotic release mechanism for controlling drive pressure from a pressurized actuator. Osmogen is contained within a small container wherein pressure built up in the osmogen in the container operates the release mechanism.

Having regard to the above, it is an object of the present invention to provide a lumen insertable capsule which enables a simple trigger mechanism to be incorporated in a less complex and inexpensive manner, and which enables simplified manufacture.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in an aspect of the invention, a capsule suitable for insertion into a lumen, such as a gastrointestinal lumen, of a human or animal subject is provided. The capsule comprises:
a capsule housing,
a reservoir configured to accommodate a drug substance, the reservoir leading to a drug outlet,
an actuation chamber,
a movable separator arranged between the actuation chamber and the reservoir wherein movement of the movable separator expels drug substance from the reservoir through the drug outlet, and
a drive system comprising a gas expansion unit and a trigger comprising a trigger member configured for engagingly operating the gas expansion unit for causing triggering of the gas expansion unit,
   wherein the gas expansion unit comprises a gas gate, the gas gate being operable from a closed state to an open state by relative movement between the gas expansion unit and the trigger member to allow pressurized gas to flow from the gas expansion unit to the actuation chamber thereby exerting load on the movable separator for expelling the drug substance, and
   wherein the trigger comprises a swellable portion comprising sponge material, wherein wetting of the sponge material causes the swellable portion to swell thereby causing relative movement between the gas expansion unit and the trigger member to open the gas gate.

Utilizing the structural properties of the sponge material enables manufacturing processes, such as handling and assembling operations, to be simplified. Additionally, due to the structural properties of the sponge, the sponge portion enables incorporation into the capsule in the form of a structural member allowing additional components to be mounted and held directly by the sponge, e.g. such as by being fixedly attached to the sponge. In addition, the structural properties of the sponge provide improved control of the relative positioning of the gas expansion unit and the trigger member prior to triggering. Further benefits of using a sponge material includes enabling the swelling to occur primarily or exclusively along a predefined direction, e.g. along the direction of relative movement between of the gas expansion unit and the trigger member.

In some forms the swellable portion is provided as a single piece of sponge material. In other forms the swellable portion is provided as multiple pieces of sponge material and/or comprising other swellable components.

In some forms of the capsule, the trigger is configured so that the trigger member exerts mechanical pressure on the gas gate upon swelling of the swellable portion thereby operating the gas gate from the closed state to the open state.

In some embodiments the capsule housing comprises a fluid inlet portion, wherein a semi-permeable membrane is arranged in the fluid inlet portion, and wherein the first surface of the swellable portion is arranged in contact with and/or adjacent to the semi-permeable membrane allowing wetting of the swellable portion by biological fluid entering through the fluid inlet portion. In certain embodiments the biological fluid is gastric fluid.

In some embodiments the semi-permeable membrane and the swellable portion are configured as an osmotic drive. In some embodiments, an osmogen or solute (e.g. volume of dry salt) is disposed in contact with the semi-permeable membrane and the swellable portion, such as in between the semi-permeable membrane and the swellable portion.

The capsule may comprise one or more openings, i.e. one or more fluid inlets, to allow a biologic fluid, such as gastric fluid, to enter the capsule for driving the trigger through osmosis.

In some forms the capsule defines a fluid inlet portion initially comprising an enteric coating adapted to dissolve when subjected to a biological fluid within the lumen, wherein biological fluid within the lumen is allowed to flow through the fluid inlet portion upon dissolution of the enteric coating. In other form, the capsule fluid inlet portion does not comprise a coating but so that fluid communication across the fluid inlet portion is enabled as soon as the capsule is inserted or ingested.

In some forms of the capsule the relative movement between the gas expansion unit and the trigger member is configured to occur along an axis and wherein the swellable portion is oriented so that, prior to swelling, the swellable portion has its largest dimension transversally, such as normally, to the axis. The sponge portion may in some embodiments be configured to expand when swelling primarily or exclusively along the axis, e.g. along a longitudinal axis of the capsule housing and or a piston sliding axis, to exert force on the trigger member for opening the gas gate. In order for providing a forceful triggering force, in various embodiments, the sponge portion may be provided so that the largest dimension of the sponge is between 2 and 8 mm, such as between 3 and 7 mm, such as between 4 and 7 mm.

In further embodiments of the capsule, the sponge material may be oriented so that prior to swelling, the sponge has its largest dimension transversally to an axis, such as the longitudinal axis and/or the piston axis. In such embodiments, manufacturing is particularly simple, as the main components of the capsule may be stacked along a single direction during assembly.

In some forms the movable separator is arranged within a cavity formed by the reservoir and wherein the movable separator is provided as a slidable piston arranged for movement along an axis within the cavity and towards the outlet. The piston may include a peripheral seal member for sealing across the piston between the actuation chamber and the reservoir. In other forms the movable separator may comprise a flexible membrane which is separating highpressure gas in the actuation chamber and the drug substance accommodated in the reservoir. The flexible membrane may divide the cavity in two separate portions wherein expansion of a first of the two separate portions by increasing gas pressure in the actuation chamber reduces volume of the other separate portion which accommodates the drug substance. In some forms the flexible membrane may be provided as a bag or similar enclosure having a single opening at the drug outlet for fluid communication through the drug outlet.

In some forms wherein the movable separator is provided as a piston configured for sliding along a piston axis within the reservoir. The piston may be formed to define an interior hollow that partly or fully accommodates the gas expansion unit.

In some forms of the capsule, the capsule housing is formed as an elongated object having its greatest dimension arranged along said axis. In other forms other shapes of the capsule housing may be provided, such as spherical, gomboc-shaped or other shapes.

In some configurations of the trigger, the swellable portion comprises a first surface and a second surface arranged opposite to the first surface, wherein the first surface is supported, during swelling of the swellable portion, relative to a structure fixedly associated with the capsule housing.

The trigger member may be configured for movement relative to the capsule housing. In such configurations the second surface is arranged for cooperation with the trigger member to cause movement of the trigger member relative to the gas expansion unit upon swelling of the swellable portion.

In some forms the the trigger member is mounted supportingly, such as being attached, relative to the swellable portion, such as directly, or indirectly via an intermediate component.

In other configurations, at least during swelling of the swellable portion, the trigger member is arranged fixedly relative to the capsule housing, wherein the gas expansion unit is configured for movement relative to the capsule housing, and wherein the second surface is arranged for cooperation with the gas expansion unit to cause movement of the gas expansion unit relative to the trigger member upon swelling of the swellable portion.

In some forms the gas gate comprises a rupturable seal, wherein when the gas gate assumes the closed state the rupturable seal seals off the gas expansion unit relative to the actuation chamber, and wherein the trigger is configured for rupturing the rupturable seal to thereby cause the gas gate to assume the open state.

In some forms the gas gate comprises a puncturable membrane, the puncturable membrane being puncturable by the trigger.

In still other forms the gas gate comprises a gas valve comprising a valve control member, the valve control member being operable by the trigger.

In some embodiments the gas expansion unit comprises a pressurized gas canister or a gas generator comprising at least one gas generating material.

Suitable materials for the sponge material may comprise one or more of the following: fibrous porous materials prepared from natural or synthetic fibers, including wool, silk, cellulose, nylon, dacron, cotton or wool felts, natural or synthetic fiber papers, or woven or knitted natural or synthetic fiber fabrics. Also, suitable materials are porous or microporous, open-celled, organic or inorganic solids such as regenerated cellulose sponge, or poly(urethane) foam.

In some forms of the capsule, the sponge material comprises biodegradable material, such as cellulose, which is adapted to degrade when subject to a liquid, but only provide substantive degradation after the swellable portion has expanded to allow the trigger to release energy from the energy source.

In applications where the lumen of the subject comprises a lumen wall the drug outlet may comprise a nozzle arrangement configured for needleless jet delivery, wherein the capsule is configured to expel drug substance through the nozzle arrangement with a penetration velocity allowing the drug substance to penetrate tissue of the lumen wall.

In other configurations the drug outlet comprises an injection needle. In still other embodiments, the drug outlet and the drive system may be configured for spraying within the lumen of the drug substance.

In exemplary embodiments, the capsule is configured for swallowing by a patient and travelling into a lumen of a gastrointestinal tract of a patient, such as the stomach, the small intestines or the large intestines. The capsule of the device may be shaped and sized to allow it to be swallowed by a subject, such as a human.

By the above arrangements an orally administered drug substance can be delivered safely and reliably into the stomach wall or intestinal wall of a living mammal subject.

The invention is further exemplified by the below clauses:

Clause 1. A capsule (100; 200; 300) suitable for insertion into a lumen, such as a gastrointestinal lumen, of a subject, wherein the capsule comprises:
a capsule housing (110, 120;210, 220; 310, 320),
a reservoir (A) configured to accommodate a drug substance, the reservoir leading to a drug outlet (190; 290; 390),
an actuation chamber (118; 218, 226; 318),
a movable separator (160; 260; 360) arranged between the actuation chamber (118; 218, 226; 318) and the reservoir (A) wherein movement of the movable separator (160; 260; 360) expels drug substance from the reservoir (A) through the drug outlet (190; 290; 390), and
a drive system comprising an energy source (150; 250; 350) and a trigger (145, 140, 170; 245, 240, 270; 345, 340, 370) operable for releasing energy from the energy source (150; 250; 260) to exert load on the movable separator (160; 260; 360) for expelling the drug substance,
wherein the trigger (145, 140, 170; 245, 240, 270; 345, 340, 370) comprises a swellable portion (140; 240; 340) comprising sponge material, wherein wetting of the sponge material causes the swellable portion to swell thereby operating the trigger (145, 140, 170; 245, 240, 270; 345, 340, 370) to release energy from the energy source (150).

Clause 2. A capsule as in clause 1, wherein the capsule housing (110, 120;210, 220; 310, 320) comprises a fluid inlet portion (115; 215; 315), a semi-permeable membrane (145; 245; 345) arranged in the fluid inlet portion, and wherein the first surface of the swellable portion (140; 240; 340) is arranged in contact with and/or adjacent to the semi-permeable membrane (145; 245; 345) allowing wetting of the swellable portion (140; 240; 340) by fluid entering through the fluid inlet portion (115; 215; 315).

Clause 3. A capsule as in clause 2, wherein the fluid inlet portion (115; 215; 315) initially comprises an enteric coating adapted to dissolve when subjected to a biological fluid within the lumen, wherein biological fluid within the lumen is allowed to flow through the fluid inlet portion (115; 215; 315) upon dissolution of the enteric coating.

Clause 4. A capsule as in any of clauses 1-3, wherein the energy source comprises a gas expansion unit (150; 250; 350) operatively coupled to the actuation chamber (118; 218, 226; 318) and the trigger (145, 140, 170; 245, 240, 270; 345, 340, 370), wherein operation of the trigger allows pressurized gas from the gas expansion unit (150; 250; 350) to increase gas pressure in the actuation chamber, and wherein the gas expansion unit (150; 250; 350) and the trigger (145, 140, 170; 245, 240, 270; 345, 340, 370) are arranged moveable relative to each other to trigger the gas expansion unit (150; 250; 350) thereby exerting load on the moveable separator for expelling of the drug substance.

Clause 5. A capsule as in clause 4, wherein the trigger (145, 140, 170; 245, 240, 270; 345, 340, 370) comprises a trigger member (170; 270; 370) configured for engagingly operating the gas expansion unit (150; 250; 350) for causing triggering of the gas expansion unit.

Clause 6. A capsule as in clause 5, wherein the swellable portion (140; 240; 340) comprises a first surface and a second surface arranged opposite to the first surface, wherein the first surface is supported, during swelling of the swellable portion (140; 240; 340), relative to a structure fixedly associated with the capsule housing.

Clause 7. A capsule as in clause 6, wherein the trigger member (170; 270; 370) is configured for movement relative to the capsule housing (110, 120;210, 220; 310, 320), and wherein the second surface is arranged for cooperation with the trigger member (170; 270; 370) to cause movement of the trigger member (170; 270; 370) relative to the gas expansion unit (150; 250; 350) upon swelling of the swellable portion.

Clause 8. A capsule as in clause 7, wherein the trigger member (170; 270; 370) is mounted supportingly relative to the swellable portion.

Clause 9. A capsule as in clause 6, wherein, during swelling of the swellable portion, the trigger member (170; 270; 370) is arranged fixedly relative to the capsule housing (110, 120;210, 220; 310, 320), wherein the gas expansion unit (150; 250; 350) is configured for movement relative to the capsule housing (110, 120;210, 220; 310, 320), and wherein the second surface is arranged for cooperation with the gas expansion unit (150; 250; 350) to cause movement of the gas expansion unit relative to the trigger member (170; 270; 370) upon swelling of the swellable portion.

Clause 10. A capsule as in any of clauses 2-9, wherein the gas expansion unit (150; 250; 350) comprises a gas gate, the gas gate being operable from a closed state to an open state by cooperating with the trigger to allow pressurized gas to flow from the gas expansion unit (150; 250; 350) to the actuation chamber.

Clause 11. A capsule as in clause 10, wherein the gas gate comprises a rupturable seal (151; 251; 351), wherein when the gas gate assumes the closed state the rupturable seal seals off the gas expansion unit (150; 250; 350) relative to the actuation chamber (118;, 218, 226; 318), and wherein the trigger is configured for rupturing the rupturable seal to thereby cause the gas gate to assume the open state.

Clause 12. A capsule as in clause 10, wherein the gas gate comprises a gas valve comprising a valve control member, the valve control member being operable by the trigger.

Clause 13. A capsule as in any of the clauses 2-13, wherein the gas expansion unit (150; 250; 350) comprises a pressurized gas canister or a gas generator comprising at least one gas generating material.

Clause 14. A capsule as in any of the clauses 1-13, wherein the sponge material (140; 240; 340) comprises biodegradable material.

Clause 15. A capsule as in any of the clauses 1-14, wherein the lumen comprises a lumen wall, wherein the drug outlet comprises a nozzle arrangement (192; 292; 392) configured for needleless jet delivery, and wherein the capsule is configured to expel drug substance through the nozzle arrangement with a penetration velocity allowing the drug substance to penetrate tissue of the lumen wall.

Clause 16. A capsule as in any of the clauses 1-15 wherein the energy source is or comprises at least one spring configured as a drive spring, wherein the spring is selected as one of a compression spring, a torsion spring, a leaf spring and a constant-force spring.

As used herein, the terms “drug”, “drug substance”, “drug product” or “payload” is meant to encompass any drug formulation capable of being delivered into or onto the specified target site. The drug may be a single drug compound, a premixed or co-formulated multiple drug compound, or even a drug product being mixed by two or more separate drug constituents wherein the mixing is performed either before or during expelling. Representative drugs include pharmaceuticals such as peptides (e.g. insulins, insulin containing drugs, GLP-1 containing drugs as well as derivatives thereof), proteins, and hormones, biologically derived or active agents, hormonal and gene-based agents, nutritional formulas and other substances in both solid, powder or liquid form. Specifically, the drug may be an insulin or a GLP-1 containing drug, this including analogues thereof as well as combinations with one or more other drugs.

### BRIEF DESCRIPTION OF THE DRAWINGS

In the following embodiments of the invention will be described with reference to the drawings, wherein

FIG. 1 is an external perspective view of an ingestible capsule 100 according to a first embodiment of the invention,

FIG. 2 is a cross-sectional side view of the ingestible capsule 100 according to the first embodiment of the invention,

FIG. 3 is a cross-sectional side view an ingestible capsule 200 according to a second embodiment of the invention,

FIG. 4 is a cross-sectional side view of an ingestible capsule 300 according to a third embodiment of the invention, and

FIG. 5 is a cross-sectional side view of an ingestible capsule 300′ according to a fourth embodiment of the invention.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The terms “assembly” and “subassembly” do not imply that the described components necessarily can be assembled to provide a unitary or functional assembly or subassembly during a given assembly procedure but is merely used to describe components grouped together as being functionally more closely related.

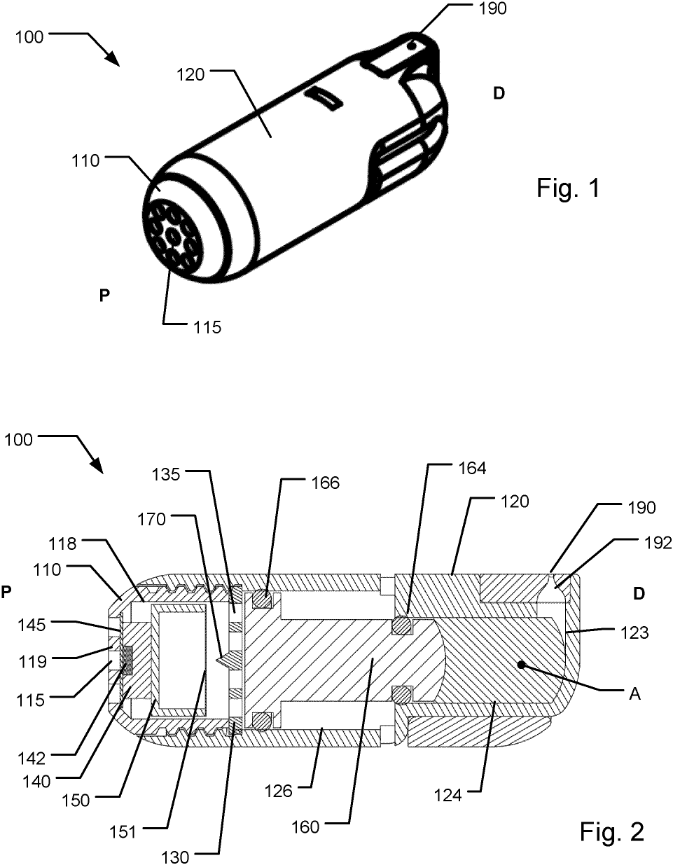

With reference to FIG. 1 a first embodiment of a drug delivery device in accordance with the invention will be described, the embodiment being designed to provide an ingestible capsule device 100 sized and shaped to be ingested by a patient and configured for subsequently being deployed when in a target lumen of the patient so as to cause a dose of a liquid drug to be expelled through a drug outlet provided at an external portion of the capsule device 100. It is to be noted that the disclosed ingestible capsule device 100, in the following referred to simply as “capsule”, is only exemplary and, in accordance with the invention, may be provided in other forms having different capsule outer shapes. Also, although the shown outlet provides an outlet nozzle opening for expelling a substance directly through the outlet, the outlet may be provided in alternative forms, such as having an outlet opening associated with an injection needle. The disclosed embodiment relates to a capsule 100 suitable for being ingested by a patient to allow the capsule to enter a lumen of the Gastro-Intestinal tract, such as the small intestines, and finally to eject a liquid dose of a payload, such as a drug substance at a target location either inside the lumen, or into tissue of the lumen wall surrounding the lumen. In other embodiments, the capsule may be configured for expelling a substance in other locations of the Gastro-Intestinal system, such as the stomach, or even in other lumen parts of a subject.

In the shown embodiment capsule 100, the substance is intended to be prepared from or provided as a single drug product. Alternatively, the substance may be prepared from at least two drug products. When the substance is prepared by two drug products, a first product may be stored within a first reservoir whereas a second product may be stored in a second reservoir and mixed prior to expelling or even mixed during expelling through the outlet. In some embodiments, the first drug component is provided initially as a lyophilized drug substance, such as a powder, whereas the second drug component is a reconstitution liquid, such as a diluent. In other embodiments, the two or more drug products are each initially provided as a liquid which are mixed with each other prior to or during drug expelling.

Referring to FIGS. 1 and 2, the capsule 100 includes a multi-part housing having an elongated shape extending along an axis, which is also referred to in the following as “the longitudinal axis”. The elongated housing includes a cylindrical section and further include rounded end portions, i.e. a proximal end portion and a distal end portion. In the shown embodiment an outlet 190 is arranged at a sidewall portion of the cylindrical section, at the distal end of the capsule 100. The outlet thus points radially outwards from a surface arranged to be in close proximity with the tissue of the lumen wall. In the shown embodiment, the capsule is shaped in shape and size to roughly correspond to a 00 elongated capsule.

The shown multi-part housing includes a first housing portion, proximal housing portion 110, arranged at the proximal end, a generally cylindrical sleeve shaped distal housing portion 120 ending at the distal end with a generally rounded end surface. In the shown embodiment the proximal and distal housing portions are fixedly mounted relative to each other by means of a threaded engagement. A proximal end wall 119 of the proximal housing portion 110 includes a multitude of openings or channels 115 serve as a fluid inlet which allows ingress of gastric fluid present in the GI tract towards the interior of the capsule 100.

FIG. 2 shows the capsule 100 in the initial state wherein the capsule is ready to be ingested by a patient. Inside capsule 100, at the distal end thereof, a hollow first cylindrical section 124 is arranged extending along the longitudinal axis and having a first diameter. The first cylindrical section 124 is terminated at the distal end by a distally arranged end face 123. The first cylindrical section 124 extends proximally towards a hollow second cylindrical section 126, coaxially arranged with the first cylindrical section 124 and having a larger diameter than the diameter of the first cylindrical section 124. A hollow third cylindrical section 118 extends coaxially with the first and second cylindrical sections 124 and 126, from the second cylindrical section to the most proximal end of the capsule 100 wherein the third cylindrical section 118 is terminated by proximal end wall 119. In the shown embodiment, proximal end wall 119 has a central planar portion.

A piston 160 is arranged for axial slideable movement inside the hollow space provided by the first cylindrical section 124 and the second cylindrical section 126. The piston 160 includes a small diameter section having a circumferential seal 164 that seals against the radially inwards surface of the first cylindrical section 124. The piston 160 further includes a large diameter section having a circumferential seal 166 that seals against the radially inwards surface of the second cylindrical section 124. The piston includes a distal facing circular end surface having a diameter which is made slightly smaller than the diameter of the first cylindrical section 124. At the proximal end of piston 160, the piston includes a proximal facing circular end surface having a diameter slightly smaller than the diameter of the second cylindrical section 126.

When the capsule assumes an initial state, i.e. prior to administration, the piston 160 is disposed in a start position remote from distally arranged end face 123. In this initial state, the circular distal end face of the piston, the radially inwards surface of the first cylindrical section 124 and the distally arranged end face 123 in combination defines a drug reservoir A. A liquid drug substance is accommodated in the reservoir A. The outlet 190 arranged at the distal end of reservoir A defines a fluid outlet passage from the reservoir to the exterior of the capsule 100. In the shown embodiment, the outlet 190 includes a jet nozzle 192 dimensioned and shaped to create a liquid jet stream of drug when the drug is forced through the outlet. In the shown embodiment, the reservoir is sealed at the outlet with a seal designed to break at high pressure of the liquid drug.

Existing jet injector systems for jet drug delivery are known in the art. A skilled person would understand how to select an appropriate jet injector that provides the correct jetting power to deliver the therapeutic substance into the lumen wall 24, for example from WO 2020/106,750 (PROGENITY INC).

In particular, the skilled person would understand that during drug delivery into a GI tract of a patient using jet injection, the jet stream created by the jet injector interfaces the lumen of the GI tract and the surface of the GI tract facing the lumen. Ultimately, the drug substance is deposited into the submucosal and/or the mucosal tissue by the substance impacting the mucosal layer of the GI tract (e.g. the epithelial layer and any mucus that may be present on the epithelial layer) as a stable jet stream of fluid with minimal breakup into a spray.

The volume of fluid of the drug substance experiences a peak fluid pressure that generates the jet stream that exits the jet injector with a peak jet velocity. The jet stream impacts the interface of the lumen of the GI tract and the surface of the GI tract facing the lumen with a peak jet power, peak jet pressure and peak jet force. The skilled person would recognise that these three parameters are interconnected.

The skilled person would understand how to assess and measure the various jet injector characteristics for suitability of use in the described type of jet injection. For example, one way to assess the jet power is to release the jets onto force sensors which measure the force the jet. Based on the force reading, and knowing the area of the nozzle and density of the jetted liquid, the jet velocity can be determined using equation 1. Based on the calculated velocity, the power (in Watts) can be calculated using equation 2. To evaluate the jet pressure (i.e. the pressure at which the jet stream is expelled), equation 3 can be used.

$$\text{F} \text{=} ρ A {V}^{2} \qquad \text{(eqation1)}$$

$$\text{P} \text{=} \frac{1}{2} ρ A {V}^{3} \qquad \text{(equation 2)}$$

$$V = \sqrt{\frac{2 ∗ {P}_{b a r} ∗ 100000}{ρ ∗ C}} \qquad \text{(equation 3)}$$

F = Force (N)
ρ= Density (kg/m3)
A= Area of nozzle (m2)
V = Velocity (m/s)
P= power (W)
P_{bar} = Pressure (bar)
C= Nozzle Loss Coefficient (Typically 0.95)

Inside capsule 100, at the proximal end thereof, a drive system is arranged configured for driving the piston 160 towards the outlet 190 upon triggering of the drive system, i.e. upon triggering by a predefined condition. The drive system comprises an energy source capable of driving forward the piston 160. The drive system is arranged inside a hollow third cylindrical section 118.

In the shown embodiment, the drive system of capsule 100 includes an energy source in the form of a gas expansion unit provided as a pre-pressurized gas cannister 150. Gas cannister 150 forms an enclosure having a cylindrical space which accommodates a gas stored at high pressure. The cylindrical space is closed by a rupturable seal 151 which in this embodiment is provided as a membrane made from a thin foil material, e.g. aluminium foil, the rupturable seal 151 facing towards the distal end of capsule 100.

In this first embodiment the gas cannister 150 is arranged axially slideable within the third cylindrical section 118. A partitioning wall 130 separates the third cylindrical section 118 and the second cylindrical section 126, the partitioning wall including a plurality of through-going apertures 135 which allow pressurized gas to flow from the third cylindrical section 118 to the second cylindrical section 126. A trigger member 170 is fixedly disposed onto the partitioning wall 130 at a central location thereof, i.e. arranged coaxially with the longitudinal axis. The trigger member 170 is formed as a pointed spike having the pointed end pointing towards the proximal direction and hence towards rupturable seal 151 of gas cannister 150.

As noted above the proximal housing portion 110, and more specifically the central planar portion of proximal end wall 119 includes a multitude of openings or channels 115 arranged at the proximal end face which allows ingress of gastric fluid into the third cylindrical section 118. A semi-permeable membrane 145 is arranged with its proximally facing surface in intimate contact with the distal facing surface of the central planar portion of proximal end wall 119. Hence, gastric fluid that enters the capsule 100 needs to pass through the opening 115 and the semi-permeable membrane 145. The central planar portion of proximal end wall 119 provides sufficient rigidity to serve as a backing for the semi-permeable membrane 145.

A piece of a sponge material 140 is arranged in proximity of the semi-permeable membrane 145. Sponge material 140 may be formed by absorbent material made from a fibrous, porous or microporous, open-celled material chosen to exhibit a marked ability to rapidly swell when being subjected to contact with a liquid. In the embodiment shown the sponge portion 140 is a dry cellulose sponge provided in compressed form, wherein the cellulose is provided as a biodegradable sponge.

The sponge portion 140 is arranged in the third cylindrical section 118 disposed axially between the semi-permeable membrane 145 and the gas cannister 150. To enable the semi-permeable membrane to quickly soak in gastric fluid through openings 115, i.e. to serve in combination with the semi-permeable membrane as an osmotic drive, a salt 142 or similar material is positioned in contact with both the semi-permeable membrane 145 and the sponge portion 140. In the shown embodiment the semi-permeable membrane 145, the sponge portion 140 and the gas canister 150 are adhered to each other in a sandwich configuration with the salt 142 arranged in a cavity formed in the sponge portion 140. For some embodiments, the sponge portion 140 may be constrained around its circumference so that the sponge primarily or exclusively expands in the axial dimension as fluid makes the sponge swell.

In the shown embodiment, the semi-permeable membrane 145, the salt 142, the sponge portion 140 and the trigger member 170 in combination forms a trigger assembly. Also, in the shown embodiment, although not visible in FIGS. 1 and 2, the openings 115 are initially covered by a pH-sensitive enteric coating which initially blocks fluid ingress through the openings 115. As known in the art, the enteric coating may be configured to utilize the marked shift in pH-level that the capsule 100 experiences when travelling from the stomach to the small intestine.

Next the operation of capsule 100 will be described. Subsequent to a patient or user swallows capsule 100, upon entering the small intestine, the enteric coating of the capsule 100 will begin dissolving and gastric fluid will soon after be available through openings 115 for the osmotic drive to provide fluid transport across the semi-permeable membrane 145.

As fluid gets into contact with sponge portion 140 the sponge rapidly starts to expand. In the shown embodiment, the sponge portion 140 may be constrained around its circumference so that the sponge primarily or exclusively expands in the axial dimension as fluid makes the sponge swell. Axial swelling of the sponge portion 140 causes the gas canister 150 to be forced to displace distally in the course of fluid ingress through the semi-permeable membrane 145. As the gas canister 150 moves distally, the trigger member 170 will start contacting the rupturable seal 151. Upon further distal movement of gas canister 150, the trigger member 170 will at some point penetrate the rupturable seal 151, whereafter the pressurized gas within gas canister will escape to the third cylindrical section 118, and due to the through-going apertures 135 of partitioning wall 130 pressurized gas will flow from the third cylindrical section 118 to the second cylindrical section 126, the two cylindrical sections 118 and 126 serving in combination as an actuation chamber. Puncture of the rupturable seal 151 will rapidly increase the gas pressure of the actuation chamber which exerts a load onto the proximal facing end surface of the piston 160. Due to the difference in cross-sectional area of the proximal facing circular end surface and the relative to the cross-sectional area of the distal facing circular end surface of the piston 160 the pressure in the actuation chamber is magnified to the hydraulic pressure in the reservoir A and the drug substance in reservoir A is thrust out through the jet nozzle 192.

Eventually, the piston 160 will bottom out relative to distally arranged end face 123 and the jet stream of drug through the jet nozzle 192 will end. After delivery of the drug substance, the capsule 100 is allowed to pass the alimentary canal and be subsequently excreted.

It is to be noticed that for other embodiments, other types of energy sources than the shown pre-pressurized gas canister may be used, such as gas expansion units relying on gas generation via a chemical reaction or phase change. In other variants, still other types of potential energy sources may be used for the energy source, such as an energized spring, e.g. a compression spring which prior to triggering is maintained in a compressed mode, and wherein a latch is operable by the trigger assembly to release the latch upon operation of the trigger causing release of the compression spring which drives forward the piston for expelling of the drug substance.

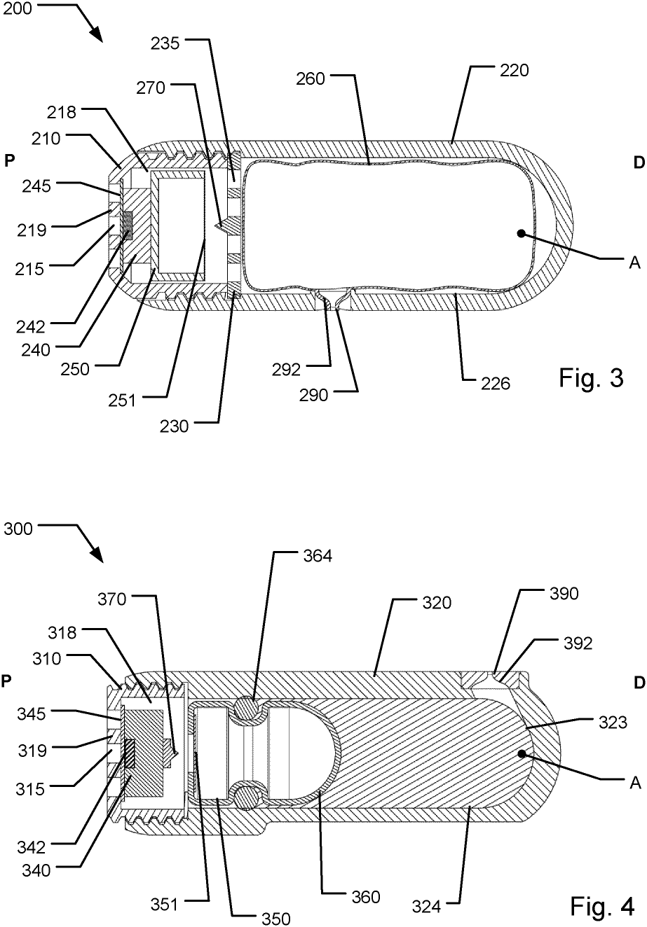

Referring now to FIG. 3, a second embodiment of a capsule 200 will now be described. The capsule 200 corresponds in many aspects to the capsule 100 but the reservoir and the expelling mechanism is different. Whereas the capsule 100 relies on a movable separator between the actuation chamber and the reservoir provide as a slidable piston 160, the capsule 200 utilizes a flexible membrane 260 separating the actuation chamber and the reservoir as a movable separator.

Capsule 200 again includes a proximal housing portion 210 and a distal housing portion 220. In capsule 200, the trigger assembly formed by semi-permeable membrane 245, sponge portion 240, salt 242 and trigger member 270 are formed similar as for the capsule 100. Also, the energy source, e.g. the gas canister 250 with rupturable seal 251 are formed similar, i.e. slidably arranged within cylindrical section 218, also referred to as “input cylindrical section”.

The outlet 290 including jet nozzle 292 is located at a side portion of the cylindrical shaped sleeve of capsule 200, arranged approximately midways between the distal end and the proximal end of the capsule 200.

A major portion of the distal housing portion 220 includes a hollow cylindrical section 226, which may be referred to “output cylindrical section” which serves both as an actuation chamber and a space for accommodating the reservoir A. A flexible gas-tight and fluid-tight membrane 260 is arranged within cylindrical section 226. The membrane forms a reservoir A, i.e. configured as a bag and forming an enclosure for the drug substance with a single opening arranged at the outlet 290.

In FIG. 3, which shows the capsule 200 in the initial state wherein the capsule is ready to be ingested by a patient, the membrane 260 assumes an expanded configuration wherein the bag defined by the membrane takes up a major portion of the cylindrical section.

Subsequent to a patient or user swallows capsule 200, upon entering the small intestine, the enteric coating of the capsule 200 will begin dissolving and gastric fluid will soon after be available through openings 215 for the osmotic drive to provide fluid transport across the semi-permeable membrane 245.

As fluid gets into contact with sponge portion 240 the sponge rapidly starts to expand. Axial swelling of the sponge portion 240 causes the gas canister 250 to be forced to displace distally in the course of fluid ingress through the semi-permeable membrane 245.

As the gas canister 250 moves distally, the trigger member 270 will start contacting the rupturable seal 251. Upon further distal movement of gas canister 250, the trigger member 270 will at some point penetrate the rupturable seal 251, whereafter the pressurized gas within gas canister will escape to the input cylindrical section 218, and due to the through-going apertures 235 of partitioning wall 230 pressurized gas will flow from the input cylindrical section 218 to the output cylindrical section 226, i.e. the actuation chamber. Puncture of the rupturable seal 251 will rapidly increase the gas pressure of the actuation chamber 226 which exerts a load onto membrane 260 causing the volume of membrane 260, i.e. the reservoir A, to become smaller. Due to the reduction in volume within the membrane 260 the hydraulic pressure in the reservoir A and the drug substance accommodated in reservoir A is thrust out through the jet nozzle 292.

Eventually, the membrane 260 will assume a collapsed configuration when the pressurized gas has evacuated substantially all of the drug substance accommodated in reservoir A and the jet stream of drug through the jet nozzle 292 will end. After delivery of the drug substance, the capsule 200 is allowed to pass the alimentary canal and be subsequently excreted.

A third embodiment of a capsule 300 in accordance with the invention is shown in FIG. 4. The capsule 300 corresponds in many aspects to the capsule 100 but the energy source is different, and the trigger assembly has been slightly modified.

Capsule 300 again includes a proximal housing portion 310 and a distal housing portion 320. In capsule 300, the trigger assembly formed by semi-permeable membrane 345, sponge portion 340, salt 342 are formed similar as for the capsule 100. However, instead of having the trigger member being arranged at a fixed position in capsule 100, the trigger member 370 of the third embodiment is mounted on the sponge 240, i.e. on a distally facing end surface of the sponge, with the spike arranged to protrude distally.

Also, the energy source, e.g. the gas canister 350 with rupturable seal 351 is formed differently compared to the first embodiment capsule 100. In capsule 300, the piston 360 is formed hollow and incorporates a gas canister 350 comprising pre-pressurized gas with gas canister as an integrated part of the piston 360. The proximal end portion of the piston 360 includes a rupturable seal 351 which is facing the pointed spike of trigger member 370.

A major portion of the distal housing portion 320 includes a hollow cylindrical section 324, which may be referred to “output cylindrical section” which serves as a space for accommodating drug substance. The proximal housing portion 310 includes a hollow cylindrical section 318, which may be referred to “input cylindrical section”, which accommodates the trigger assembly and which serves as an actuation chamber. When the capsule assumes the initial state, i.e. prior to administration, the piston 360 is disposed in a start position remote from distally arranged end face 323. In this initial state, the circular distal end face of the piston, the radially inwards surface of the output cylindrical section 324 and the distally arranged end face 323 in combination defines a drug reservoir A. A liquid drug substance is accommodated in the reservoir A. The outlet 390 arranged at the distal end of reservoir A defines a fluid outlet passage from the reservoir to the exterior of the capsule 300. Also in this shown embodiment, the outlet 390 includes a jet nozzle 392 dimensioned and shaped to create a liquid jet stream of drug when the drug is forced through the outlet. The reservoir is sealed at the outlet with a seal designed to break at high pressure of the liquid drug.

In FIG. 4 the capsule 300 is shown in the initial state wherein the capsule is ready for ingestion by a patient. Subsequent to a patient or user swallows capsule 300, upon entering the small intestine, the enteric coating of the capsule 200 will begin dissolving and gastric fluid will soon after be available through openings 315 for the osmotic drive to provide fluid transport across the semi-permeable membrane 345.

As fluid gets into contact with sponge portion 340 the sponge rapidly starts to expand. Axial swelling of the sponge portion 340 causes the trigger member 370 to be forced distally in the course of fluid ingress through the semi-permeable membrane 345 into the input cylindrical section 318.

When moved distally, the trigger member 370 will start contacting the rupturable seal 351. Due to the incompressibility of the drug substance, the piston 360 will initially maintain its axial position inside output cylindrical section 324. Upon further distal movement the trigger member 370, it will at some point penetrate the rupturable seal 351 of gas canister 350, whereafter the pressurized gas within gas canister will escape to the input cylindrical section 318, i.e. the actuation chamber. Puncture of the rupturable seal 251 will rapidly increase the gas pressure of the actuation chamber which exerts a load onto the piston 360 causing the piston to moved distally and causing the volume of the reservoir A to be reduced. Due to the reduction in volume of the reservoir A the drug substance accommodated in the reservoir will be thrust out through the jet nozzle 292.

Eventually, the piston 360 will bottom out relative to distally arranged end face 323 and the jet stream of drug through the jet nozzle 392 will end. After delivery of the drug substance, the capsule 300 is allowed to pass the alimentary canal and be subsequently excreted.

Referring to the capsules as shown in FIGS. 2 through 4, during assembly of the capsules, a particularly simple assembly procedure may be provided due to the sponge material being easily handled, but also due to the structural properties of the sponge portion which allows the sponge to provide as a structural member allowing mounting of additional components directly to the sponge to be performed, such as the semi-permeable membrane and/or the trigger member.

Whereas the capsule 100, 200 and 300 utilizes a gas gate in form of a rupturable membrane, wherein a trigger member performs as a spike for puncturing the rupturable seal of the gas canister, other trigger members relying on different principles for opening a gas gate may be utilized. For example, the sponge portion may in itself provide as a trigger member and be arranged to exert a force directly to a breakable membrane which simply breaks upon the sponge material forcing its way through the breakable membrane. Also, a gas gate may be provided by means of a gas valve comprising a valve control member which is being operated by the trigger member for opening the valve control member, which allows pressurized gas to increase gas pressure in the actuation chamber.

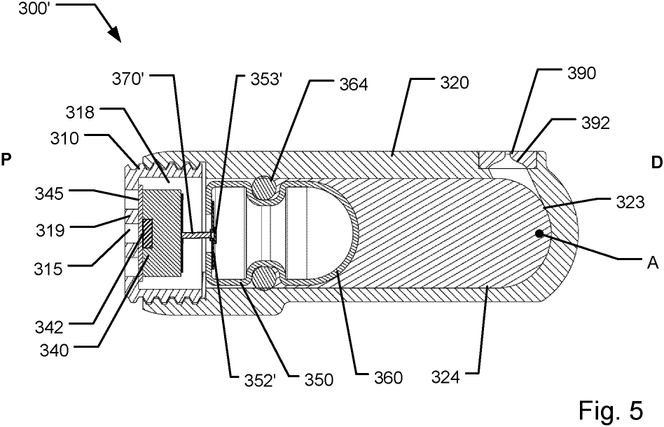

Referring to an exemplary gas gate embodiment, FIG. 5 shows schematically a fourth embodiment capsule 300′ according to the invention which in most aspects is formed similarly to the third embodiment capsule 300 discussed above. However, instead of the spike 370 and rupturable seal 351 configuration of the capsule 300, the fourth embodiment capsule 300′ includes a gas gate formed by a valve system 352′/353′ which is configured to cooperate with trigger member 370′. The exemplary valve system shown includes a first valve member 352′ essentially forming a thin wall surface closing off a proximally facing opening of gas canister, i.e. at the proximal end portion of the piston 360. A valve control member 353′ is shown cooperating with a valve seat portion of first valve member 352′ so as to seal the gas canister during storage. In the shown embodiment, the first valve member 352′ and the valve control member

The valve control member 353′, when exerted to distal directed force from the trigger member 370′, is configured to become moved distally relative to the seat portion of first valve member 352′ allowing pressurized gas to escape from the gas canister 350 into the input cylindrical section 318, i.e. the actuation chamber.

The trigger member 370′ of the fourth embodiment is again mounted on a distally facing end surface of the sponge, with the spike member being formed to include a push rod configured to exert a distally directed force on the valve control member 353′. Upon expansion of the sponge portion 340, when wetted by intestinal fluid, the trigger member 370′ will start to push valve control member 353′ distally relative to first valve member 352′ so that the valve control member is either pushed away and separated completely from the first valve member 352′ or held permanently by a retention mechanism (non-referenced) in the open position relative to the first valve member 352′. In the open position pressurized gas is allowed to flow unhindered from the gas canister to the actuation chamber.

Still, in accordance with the present invention, it is to be noted that the shown valve system 352′/353′ is only exemplary and that alternative embodiments in accordance with the present invention may incorporate other valve configurations.

As described in the above embodiments, subsequent to swallowing, the capsule device first moves through the stomach and subsequently enters the small intestine. Due to the enteric coating becomes dissolved when entering the small intestine the osmotic drive will only be initiated upon the enteric coating becoming sufficiently dissolved for fluid ingress through the fluid inlet is enabled.

An enteric coating may be any suitable coating that allows the coated object to be released in the intestine. In some cases, an enteric coating may dissolve preferentially in the small intestine as compared to the stomach. In other embodiments, the enteric coating may hydrolyse preferentially in the small intestine as compared to the stomach. Non-limiting examples of materials used as enteric coatings include methyl acrylate-methacrylic acid copolymers, cellulose acetate succinate, hydroxy propyl methyl cellulose phthalate, hydroxy propyl methyl cellulose acetate succinate (i.e., hypromellose acetate succinate), polyvinyl acetate phthalate (PVAP), methyl methacrylate-methacrylic acid copolymers, and sodium alginate, and stearic acid. Additional examples are disclosed in e.g. US 2018/0193621 hereby incorporated by reference. A given object (here: capsule), or a fluid inlet only, may be coated with an enteric coating. The enteric coating may be composed to be soluble at a given pH or within a given pH range, e.g. at a pH greater than 5.5, at a pH greater than 6.5, within a range of about 5.6 to 6 or within a range of about 5.6 to 6.5 or 7. The dissolution time at an intestinal pH may be controlled or adjusted by the composition of the enteric coating. For example, the dissolution time at an intestinal pH may be controlled or adjusted by the thickness of the enteric coating.

In other embodiments, the condition for controlling when triggering is to occur may be provided by means of other principles. For example, a dissolvable layer may be disposed initially blocking the fluid inlet of the capsule, with dissolution of the dissolvable layer being initiate at first exposure to gastric fluid, with the timing of the dissolvable layer being decisive for the location at which the capsule deploys. Also, such as for a stomach deployable capsule, no coating may be present, so that the triggering of the capsule occurs as soon as sufficient liquid has been transferred through the semi-permeable membrane. Still other triggering principles may rely on temperature change induced passage of gastric fluid though the fluid inlet and into the capsule triggering assembly.

Although the above description of exemplary embodiments mainly concern ingestible capsules for delivery in the small intestines, the present invention generally finds utility in capsule devices for lumen insertion in general, wherein a capsule device is positioned into a body lumen for delivery of a drug product. Non-limiting examples of capsule devices include capsule devices for delivery in the stomach or delivery into the tissue of the stomach wall. For example, various self-righting or self-orienting structures and/or methods described in WO 2018/213600 can be employed by the capsule device in accordance with the present disclosure. WO 2018/213600 is incorporated herein by reference in its entirety.

Drug delivery may be performed using a delivery member, such as a needle, via a jet stream of liquid to provide liquid jet penetration into the mucosal lining or via spraying inside the lumen. In still other embodiments, the inventive trigger principle set forth in this disclosure may be used to trigger delivery of a solid drug pellet which is to be inserted into a lumen wall.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A capsule suitable for insertion into a lumen, such as a gastrointestinal lumen, of a subject, wherein the capsule comprises:
a capsule housing,
a reservoir configured to accommodate a drug substance, the reservoir leading to a drug outlet,
an actuation chamber,
a movable separator arranged between the actuation chamber and the reservoir wherein movement of the movable separator expels drug substance from the reservoir through the drug outlet, and
a drive system comprising a gas expansion unit and a trigger comprising a trigger member configured for engagingly operating the gas expansion unit for causing triggering of the gas expansion unit,
wherein the gas expansion unit comprises a gas gate, the gas gate being operable from a closed state to an open state by relative movement between the gas expansion unit and the trigger member to allow pressurized gas to flow from the gas expansion unit to the actuation chamber thereby exerting load on the movable separator for expelling the drug substance, and
wherein the trigger comprises a swellable portion comprising sponge material, wherein wetting of the sponge material causes the swellable portion to swell thereby causing relative movement between the gas expansion unit and the trigger member to open the gas gate.

2. A capsule as in claim 1, wherein the capsule housing comprises a fluid inlet portion, wherein a semi-permeable membrane is arranged in the fluid inlet portion, and wherein the first surface of the swellable portion is arranged in contact with and/or adjacent to the semi-permeable membrane allowing wetting of the swellable portion by biological fluid entering through the fluid inlet portion.

3. A capsule as in claim 2, wherein the fluid inlet portion initially comprises an enteric coating adapted to dissolve when subjected to a biological fluid within the lumen, wherein biological fluid within the lumen is allowed to flow through the fluid inlet portion upon dissolution of the enteric coating.

4. A capsule as in claim 1, wherein said relative movement between the gas expansion unit and the trigger member occurs along an axis and wherein the swellable portion is oriented so that, prior to swelling, the swellable portion has its largest dimension transversally to the axis.

5. A capsule as in claim 4, wherein the movable separator is provided as a piston configured for sliding within the reservoir along said axis.

6. A capsule as in claim 4, wherein the capsule housing is formed as an elongated object having its greatest dimension arranged along said axis.

7. A capsule as in claim 1, wherein the swellable portion comprises a first surface and a second surface arranged opposite to the first surface, wherein the first surface is supported, during swelling of the swellable portion, relative to a structure fixedly associated with the capsule housing.

8. A capsule as in claim 7, wherein the trigger member is configured for movement relative to the capsule housing, and wherein the second surface is arranged for cooperation with the trigger member to cause movement of the trigger member relative to the gas expansion unit upon swelling of the swellable portion.

9. A capsule as in claim 8, wherein the trigger member is mounted supportingly relative to the swellable portion.

10. A capsule as in claim 7, wherein, during swelling of the swellable portion, the trigger member is arranged fixedly relative to the capsule housing, wherein the gas expansion unit is configured for movement relative to the capsule housing, and wherein the second surface is arranged for cooperation with the gas expansion unit to cause movement of the gas expansion unit relative to the trigger member upon swelling of the swellable portion.

11. A capsule as in claim 1, wherein the gas gate comprises a rupturable seal, wherein when the gas gate assumes the closed state the rupturable seal seals off the gas expansion unit relative to the actuation chamber, and wherein the trigger is configured for rupturing the rupturable seal to thereby cause the gas gate to assume the open state.

12. A capsule as in claim 11, wherein the gas gate comprises a gas valve comprising a valve control member, the valve control member being operable by the trigger member.

13. A capsule as in claim 1, wherein the gas expansion unit comprises a pressurized gas canister or a gas generator comprising at least one gas generating material.

14. A capsule as in claim 1, wherein the sponge material comprises biodegradable material.

15. A capsule as in claim 1, wherein the capsule is suitable for insertion into a lumen having a lumen wall, wherein the drug outlet comprises a nozzle arrangement configured for needleless jet delivery, and wherein the capsule is configured to expel drug substance through the nozzle arrangement with a penetration velocity allowing the drug substance to penetrate tissue of the lumen wall.
